(19) United States
(12) Patent Application Publication
(10) Pub. No.: US 2010/0160763 A1
(43) Pub. Date: Jun. 24, 2010

(54) ANTI-INTERFERENCE PHYSIOLOGICAL SENSING DEVICE

(76) Inventors: Dung-Han Tsai, Taipei County (TW); Hsin-Yi Pai, Taipei County (TW)

Correspondence Address: NORTH AMERICA INTELLECTUAL PROPERTY CORPORATION, P.O. BOX 506, MERRIFIELD, VA, 22116, US

(21) Appl. No.: 12/435,420

(22) Filed: May 5, 2009

(30) Foreign Application Priority Data
Dec. 24, 2008 (TW) 097150409

(51) Int. Cl. A61B 5/04; A61B 5/00

(52) U.S. Cl. 600/393

(57) ABSTRACT

An anti-interference physiological sensing device includes an electrode layer for detecting a physiological signal of an examinee, and an insulating layer disposed on a side of the electrode layer for insulating the electrode layer from static electricity interference.

### BACKGROUND OF THE INVENTION

1. Field of the Invention

The present invention relates to an anti-interference physiological sensing device, and more particularly, to an anti-interference physiological sensing device capable of preventing static electricity interference.

2. Description of the Prior Art

In an advanced medical technology, physiological sensing devices are main products in a medical appliances market. For example, the physiological sensing devices capable of detecting an electrocardiogram, breath signal, or body action are popularized. However, the conventional physiological sensing devices are immobile, and an examinee has to take physical examination at the fixed place. For solving this problem of inconvenience, a wearable physiological sensing device is developed for being equipped on the examinee to detect physiological signals of the examinee on the go. Please refer to FIG. 1. FIG. 1 is a diagram of a wearable physiological sensing device 10 in the prior art. The wearable physiological sensing device 10 includes an electrode layer 12 whereon at least one fabric electrode 14 is disposed for contacting the examinee's skin so as to detect physiological signals of the examinee. The wearable physiological sensing device 10 further includes an elastic fabric layer 18 combined with the electrode layer 12 and contacting with clothes 20 possibly. When the wearable physiological sensing device 10 is utilized for a long period in daily life, friction of the clothes 20 and the elastic fabric layer 18 causes static electricity interference to the electrode layer 12 so that the detected physiological signals are interfered by static electricity noise. It reduces the reliability and the precision of the wearable physiological sensing device.

### SUMMARY OF THE INVENTION

According to the claimed invention, an anti-interference physiological sensing device includes an electrode layer for detecting a physiological signal of an examinee, and an insulating layer disposed on a side of the electrode layer for insulating the electrode layer from static electricity interference.

According to the claimed invention, at least one fabric electrode is disposed on the electrode layer for contacting the examinee's skin so as to detect the physiological signal of the examinee.

According to the claimed invention, the anti-interference physiological sensing device further includes an elastic fabric layer disposed outside the insulating layer.

According to the claimed invention, the insulating layer is disposed between the electrode layer and the elastic fabric layer in a sewing, thermo-compression, or gluing manner.

According to the claimed invention, the insulating layer is coated on an inner side of the elastic fabric layer.

According to the claimed invention, the elastic fabric layer is made of synthetic fiber material.

According to the claimed invention, the electrode layer is for detecting an electrocardiogram signal, a breath signal, a body resistance, or change of body temperature of an examinee.

According to the claimed invention, the insulating layer is coated on the side of the electrode layer.

According to the claimed invention, the insulating layer is made of plastic material, insulating gum, rubber, silicon gel, insulating cloth material, cotton material, cloth bakelite material, polyester fiber material, glass fiber material, asbestos material, wool material, Teflon material, or mica material.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a diagram of a wearable physiological sensing device in the prior art.

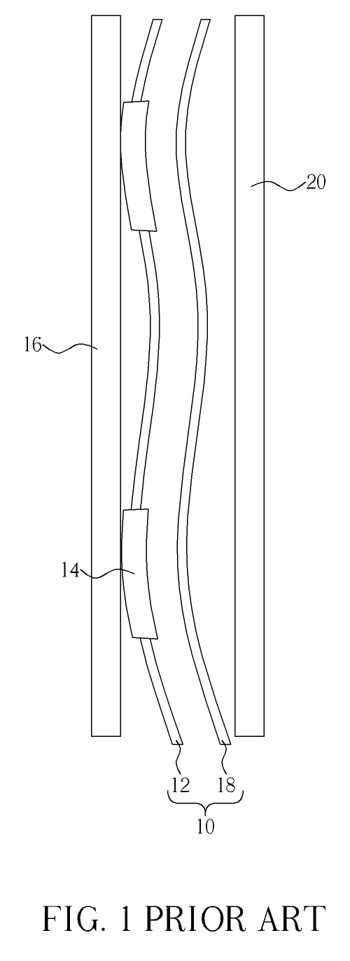

FIG. 2 is a perspective drawing of an anti-interference physiological sensing device according to a preferred embodiment of the present invention.

FIG. 3 is a diagram of the anti-interference physiological sensing device according to the preferred embodiment of the present invention.

FIG. 4 is a diagram of an anti-interference physiological sensing device according to another embodiment of the present invention.

### DETAILED DESCRIPTION

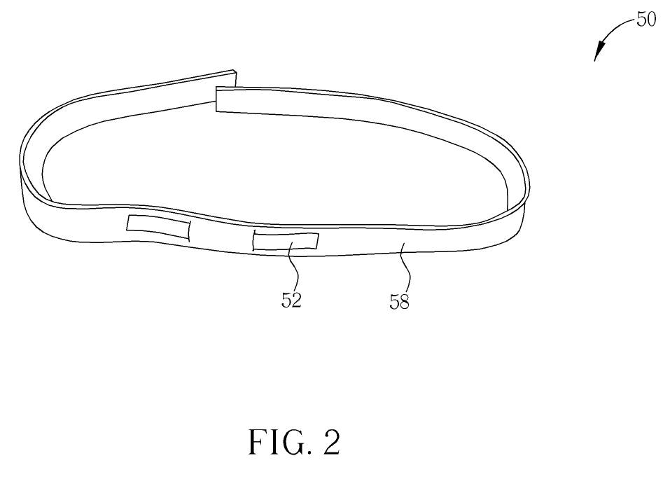

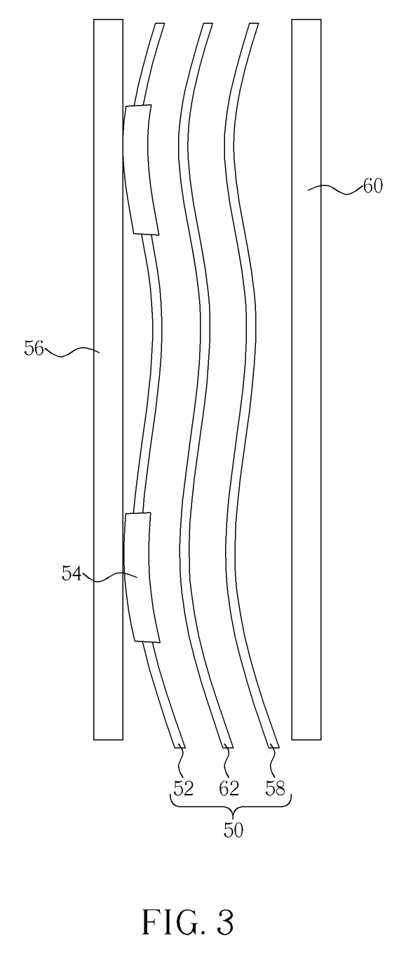

Please refer to FIG. 2 and FIG. 3. FIG. 2 is a perspective drawing of an anti-interference physiological sensing device 50 according to a preferred embodiment of the present invention. FIG. 3 is a diagram of the anti-interference physiological sensing device 50 according to the preferred embodiment of the present invention. The anti-interference physiological sensing device 50 can be a wearable physiological sensing device. The anti-interference physiological sensing device 50 can be a belt for detecting rhythm of the heart, such as heart rate, an electrocardiogram signal, a breath signal, a body resistance, or change of body temperature of an examinee. The anti-interference physiological sensing device 50 can be applied for detection of exercise heart rate, electrocardiogram of a patient with heart disease, and so on. The anti-interference physiological sensing device 50 includes an electrode layer 52 whereon at least one fabric electrode 54 is disposed for contacting the examinee's skin 56 so as to detect a physiological signal of the examinee, such as an electrocardiogram signal, a breath signal, a body resistance, or change of body temperature of the examinee. The fabric electrode 54 can be flexible conductive cloth. The fabric electrode 54 can be woven into other material of the electrode layer 52 as conductive fibers or sewed on the electrode layer 52. The anti-interference physiological sensing device 50 further includes an elastic fabric layer 58 contacting with clothes 60 possibly. The elastic fabric layer 58 can be made of elastic cloth material, synthetic fiber material, or compound of synthetic fibers and cotton, such as Nylon or Lycra.

The anti-interference physiological sensing device 50 further includes an insulating layer 62 disposed between the electrode layer 52 and the elastic fabric layer 58. The insulating layer 62 is made of insulating material for insulating the electrode layer 52 from static electricity interference generated due to friction of the elastic fabric layer 58 and the clothes 60. The insulating layer 62 can be disposed between the electrode layer 52 and the elastic fabric layer 58 in a sewing, thermo-compression, or gluing manner. The insulating layer 62 also can be coated on an inner side of the electrode layer 52 or on an inner side of the elastic fabric layer 58. An area of the insulating layer 62 is larger than a conductive area of the electrode layer 52. The structure of disposition of the insulating layer 62 between the electrode layer 52 and the elastic fabric layer 58 for insulating static electricity generated due to friction of the elastic fabric layer 58 and the clothes 60 is within the scope of the present invention. The elastic fabric layer 58 can be selectively disposed, that is, the insulating layer 62 can be disposed on an outer side of the electrode layer 52 so as to insulate the electrode layer 52 from outside static electricity interference, and the elastic fabric layer 58 is omitted as the anti-interference physiological sensing device for exercise purpose.

In addition, the insulating layer 62 can be made of plastic material, insulating gum, rubber, silicon gel, glass fiber material, asbestos material, wool material, Teflon material, mica material, diesel paper, NIKOLYTE, Mylar, polyester film, polyester fiber, and so on. The insulating layer 62 also can be made of insulating cloth material, such as cotton material, cloth bakelite material, and so on.

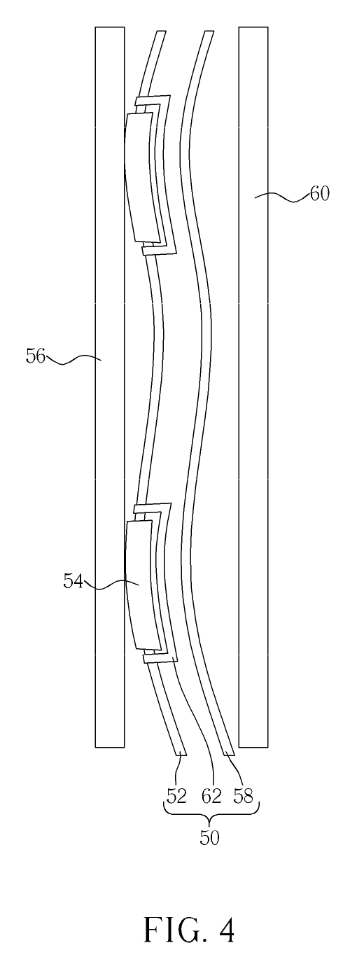

Please refer to FIG. 4. FIG. 4 is a diagram of an anti-interference physiological sensing device 100 according to another embodiment of the present invention. The difference between this embodiment and the above-mentioned embodiment is that the insulating layer 62 is disposed between the electrode layer 52 and the elastic fabric layer 58 partly. The insulating layer 62 is also capable of insulating static electricity generated due to friction of the elastic fabric layer 58 and the clothes 60. The insulating layer 62 is just utilized for covering and insulating the fabric electrodes 54 on the electrode layer 52 from the elastic fabric layer 58 so as to prevent static electricity generated due to friction of the elastic fabric layer 58 and the clothes 60 from conducting to the fabric electrodes 54 on the electrode layer 52. It can economize material of the insulating layer 62.

In conclusion, the insulating layer 62 made of insulating material is disposed outside the electrode layer 52 or between the electrode layer 52 and the elastic fabric layer 58 for insulating the electrode layer 52 from static electricity interference generated due to friction of the elastic fabric layer 58 and the clothes 60 or outside static electricity interference so as to prevent interference of the detected physiological signals. It can increase the reliability and the precision of the physiological sensing device. For example, it can increase the reliability of heart rate calculation or the precision of analysis software in the post processing for upgrading value of clinical diagnosis. Besides, the present invention has commercial competitiveness due to low cost and simplified manufacture.

In contrast to the prior art, the present invention utilizes the insulating layer disposed on a side of the electrode layer for insulating the electrode layer from static electricity interference generated due to friction of the elastic fabric layer and the clothes or outside static electricity interference so as to prevent interference of the detected physiological signals. It can increase the reliability and the precision of the physiological sensing device.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. An anti-interference physiological sensing device comprising:
an electrode layer for detecting a physiological signal of an examinee; and
an insulating layer disposed on a side of the electrode layer for insulating the electrode layer from static electricity interference.

2. The anti-interference physiological sensing device of claim 1 wherein at least one fabric electrode is disposed on the electrode layer for contacting the examinee's skin so as to detect the physiological signal of the examinee.

3. The anti-interference physiological sensing device of claim 1 further comprising an elastic fabric layer disposed outside the insulating layer.

4. The anti-interference physiological sensing device of claim 3 wherein the insulating layer is disposed between the electrode layer and the elastic fabric layer in a sewing, thermo-compression, or gluing manner.

5. The anti-interference physiological sensing device of claim 3 wherein the insulating layer is coated on an inner side of the elastic fabric layer.

6. The anti-interference physiological sensing device of claim 3 wherein the elastic fabric layer is made of synthetic fiber material.

7. The anti-interference physiological sensing device of claim 3 wherein the electrode layer is for detecting an electrocardiogram signal, a breath signal, a body resistance, or change of body temperature of an examinee.

8. The anti-interference physiological sensing device of claim 1 wherein the insulating layer is coated on the side of the electrode layer.

9. The anti-interference physiological sensing device of claim 3 wherein the insulating layer is made of plastic material.

10. The anti-interference physiological sensing device of claim 1 wherein the insulating layer is made of insulating gum.

11. The anti-interference physiological sensing device of claim 1 wherein the insulating layer is made of rubber.

12. The anti-interference physiological sensing device of claim 1 wherein the insulating layer is made of silicon gel.

13. The anti-interference physiological sensing device of claim 1 wherein the insulating layer is made of insulating cloth material.

14. The anti-interference physiological sensing device of claim 13 wherein the insulating layer is made of cotton material.

15. The anti-interference physiological sensing device of claim 13 wherein the insulating layer is made of cloth bakelite material.

16. The anti-interference physiological sensing device of claim 1 wherein the insulating layer is made of polyester fiber material.

17. The anti-interference physiological sensing device of claim 1 wherein the insulating layer is made of glass fiber material.

18. The anti-interference physiological sensing device of claim 1 wherein the insulating layer is made of asbestos material.

19. The anti-interference physiological sensing device of claim 1 wherein the insulating layer is made of wool material.

20. The anti-interference physiological sensing device of claim 1 wherein the insulating layer is made of Teflon material.

21. The anti-interference physiological sensing device of claim 1 wherein the insulating layer is made of mica material.
